(19) United States
(12) Patent Application Publication
(10) Pub. No.: US 2009/0169589 A1
(43) Pub. Date: Jul. 2, 2009

(54) FILLER PIGMENTS

(76) Inventors: Christoph Schmidt, Kriftel (DE); Sabine Schoen, Herten (DE); Tamio Noguchi, Iwaki-Shi (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/335,681

(22) Filed: Dec. 16, 2008

(30) Foreign Application Priority Data
Dec. 17, 2007 (EP) 07024443.9

(51) Int. Cl. A61K 8/19; C09B 67/22; C09D 11/02

(52) U.S. Cl. 424/401; 106/461; 106/417; 106/449; 106/459; 106/450; 106/31.65; 523/200

(57) ABSTRACT

Filler pigments based on platelet-shaped substrates, which are coated with barium sulfate and at least two metal oxides and/or metal hydroxides are highly suitable as filler pigments especially for cosmetic formulations.

This invention relates to filler pigments based on platelet shaped substrates which are coated with a layer containing barium sulfate and at least two metal oxides and/or metal hydroxides. These filler pigments are highly suitable in cosmetic applications.

It is known to use flaky powders, such as, for example, mica, e.g. muscovite or sericite, or clay materials, such as kaolin or talc, as these are starting materials for the preparation of filler powders which are especially useful in cosmetics. These filler powders by themselves do not possess the necessary properties, i.e., adhesion and extension, for the use in diverse kinds of cosmetics like face powders, make-ups, and the like. Therefore, they are conventionally mixed with additives, like titanium dioxide, metal soaps and/or calcium carbonate.

EP 0 142 695 B1 discloses pigments based on mica coated with barium sulfate. These pigments are suitable for the use in foundations like face powders, etc., due to their tactile and optical properties. However, these filler pigments have particular disadvantages like photoactivity and whitish appearance.

The object of the present invention was to provide a photostable filler pigment based on platelet-shaped substrates which shows no gloss, provides good skin-feeling and natural look.

Surprisingly, filler pigments with less or no photoactivity have now been found, which, besides an extraordinarily soft skin feeling, have a good spreadability (i.e., incorporation and homogenization into the application medium) in applications and show a hiding characteristic which supports natural appearance of the skin. This invention therefore relates to filler pigments, which are characterized in that a platelet-shaped substrate is coated with barium sulfate and at least two metal oxides and/or metal hydroxides.

The invention also relates to a process for the preparation of a flaky filler pigment. Owing to the advantageous properties, the filler pigments according to the invention are universally suitable for a large number of very different applications. The present invention accordingly also relates to the use of these filler pigments in cosmetics, paints, coatings, plastics, films, free-flowing preparations and dry preparations like granules, pellets, etc.

The invention likewise relates to cosmetic formulations, such as, for example, make-ups, compact-powders, loose powders, lipsticks, etc. which comprise the filler pigment according to the invention. The filler pigment is highly suitable as light-diffusing pigment which means it has a balanced relation of transparency, scattering and reflection. Since it scatters light to minimize the visibility of wrinkles, the filler pigment is highly useful for make-ups, anti-wrinkle products and skin correctors.

The filler pigments according to the invention are based on flake-form substrates. Suitable substrates are flakes of natural or synthetic mica, phyllosilicates, silicon dioxide, tin dioxide, zirconium dioxide, glass, aluminium oxide, titanium dioxide, magnesium fluoride and/or iron oxide or mixtures thereof. The substrate of the filler pigment according to the invention is preferably mica (synthetic and natural) flakes, Al₂O₃ flakes, glass flakes, talc, kaolin, SiO₂ flakes, and most preferably mica.

Preferred SiO₂ flakes have a uniform layer thickness and are preferably produced in accordance with the International patent application WO 93/08237 on a continuous belt by solidification and hydrolysis of a water-glass solution. “Uniform layer thickness” here is taken to mean a layer thickness tolerance of from 3 to 10%, preferably from 3 to 6%, of the total dry layer thickness of the particles. The flake-form silicon dioxide particles are generally in amorphous form.

Glass flakes are preferred owing to their smooth surfaces and high transparence. The size of the base substrates is not crucial per se and can be matched to the particular application. Particular preference is given to glass flakes having an average thickness of <2 μm. Thicker flakes generally cannot be employed in common printing processes and in demanding paint finishes. Thin glass flakes provide a better skin feeling compared to thick flakes having thicknesses >1 μm. Therefore, the glass flakes preferably have thicknesses of <1 μm, in particular of <0.9 μm, very particularly preferably of <0.7 μm. Particular preference is given to glass flakes having thicknesses of 0.25-0.7 μm. The diameter of the glass flakes is preferably 20-200 μm, particularly preferably 10-60 μm, and most preferably ≦40 μm. Glass flakes having these dimensions are commercially available and/or can be produced by known processes, such as, for example, tube blowing (Nippon Sheet Glass), spinning process (Glassflake Ltd.)

Preferred Al₂O₃ flakes may be doped or undoped. Suitable Al₂O₃ flakes are those for example which are disclosed in JP 3242561 B.

The diameter of the substrates is usually below 100 μm, preferably below 50 and most preferably ≦40 μm. The thickness is from 50-2000 nm, preferably 50-1000 nm and particularly preferably 50-500 nm.

The average aspect ratio of the flake-form substrates, i.e. the ratio of the average length measurement value, which corresponds to the average diameter here, to the average thickness measurement value, is usually from 2 to 2000, preferably from 2 to 1000 and particularly preferably from 2 to 200.

Before application of the barium sulfate layer and/or the metal oxide layers on the substrate, a thin dielectric layer where 1.4<n<2.7 (n=refractive index), can optionally also be deposited. A coating of this type, for example on glass flakes, can consist, for example of a SiO₂ layer or ZnO layer, preferably of a SiO₂ layer with a thickness of 2-20 nm.

The filler pigments according to the invention contain 5-200 wt.-%, preferably 5-100 wt.-% and most preferably 10-50 wt.-% of barium sulfate based on the substrate.

The barium sulfate layer is combined with at least two metal oxides. The weight ratio of the barium sulfate and the metal oxides may vary from 1:10 to 5:1 depending on the desired properties, e.g. the hiding power and color of the filler powder.

In the case that the barium sulfate layer contains titanium dioxide the titanium dioxide can be in the anatase or rutile modification. Rutile is the preferred modification. The rutile layer can be prepared according to the process described in EP 0 271 767.

Preferred metal oxides/hydroxides are selected from TiO₂, SnO₂, ZnO, Fe₂O₃, Fe₃O₄, SiO₂, Al₂O₃, ZrO₂ and the corresponding hydroxides as well as combinations thereof.

In addition the barium sulfate layer may also be doped with carbon black and/or organic or inorganic colorants, where the proportion of doping should not exceed 10% by weight, based on the BaSO₄ layer.

In a preferred embodiment, additionally the filler pigment can be coated with a colorant selected from the group of Carmine red, Prussian blue, indanthrene brilliant rosa, 1,4-diketo-pyrrolopyrrol derivatives, derivatives of thioindigo-, indigo-, triphenylmethane-, azo-, anthrachinone-, phthalocyanine- or indanthrene, Fe₂O₃, Cr₂O₃, BiVO₄, CoAl₂O₄ or Fe₃O₄.

If the TiO₂ layer essentially consists of rutile, full-area or partial coating with SnO₂ nuclei is preferably carried out before the coating with TiO₂. This very thin SnO₂ layer has maximum thicknesses of 20 nm, preferably ≦10 nm, most preferably ≦5 nm. The SnO₂ layer may also be distributed on the surface or the substrate as simple dots.

The photostability of the filler pigments can be increased with a SiO₂ layer on the surface of the final filler pigment. These pigments have the further advantage that they do not show any or less reactions with dihydroxy acetone (DHA) which is often used in self-tanning cosmetics.

The base substrate can be coated with a mixed layer containing the barium sulfate and at least two metal oxides. Furthermore, it is possible to apply the metal oxide layer(s) on the base substrate first and secondly the barium sulfate layer or the barium sulfate layer on the base substrate and secondly the metal oxide layer(s). The metal oxide layer consists of at least one metal oxide. The metal oxide layer can be a mixed metal oxide layer consisting of at least two different metal oxides or of two layers of different metal oxides. In general, any order of the oxide layers and the barium sulfate is possible.

Preferred filler pigments have the following layer structures:

Metal oxide 1 and metal oxide 2 can be the same or different. In a preferred embodiment metal oxide 1 and metal oxide 2 are different.

| | |
|---|---|
| substrate | |
| 1^{st} layer: | BaSO₄ |
| 2^{nd} layer: | metal oxide 1 |
| 3^{rd} layer: | metal oxide 2 or |
| substrate | |
| 1^{st} layer: | metal oxide 1 |
| 2^{nd} layer: | BaSO₄ |
| 3^{rd} layer: | metal oxide 2 or |
| substrate | |
| 1^{st} layer: | metal oxide 1 |
| 2^{nd} layer: | metal oxide 2 |
| 3^{rd} layer: | BaSO₄ or |
| substrate | |
| 1^{st} layer: | mixture of BaSO₄ + metal oxide 1 |
| 2^{nd} layer: | metal oxide 2 or |
| substrate | |
| 1^{st} layer: | metal oxide 1 |
| 2^{nd} layer: | mixture of BaSO₄ + metal oxide 2 or |
| substrate | |
| 1^{st} layer: | mixture of BaSO₄, metal oxide 1 and metal oxide 2. |

Especially preferred filler pigments have the following layer structures:

substrate+SnO₂+TiO₂+BaSO₄
substrate+mixture of SnO₂+TiO₂+BaSO₄
substrate+mixture of SnO₂+TiO₂+BaSO₄+SiO₂
substrate+SiO₂+TiO₂+BaSO₄
substrate+SiO₂+SnO₂+BaSO₄
substrate+SnO₂+TiO₂+BaSO₄+Carmine red
substrate+SnO₂+TiO₂+BaSO₄+Prussian Blue
substrate+SnO₂+BaSO₄/TiO₂
substrate+SnO₂+TiO₂+BaSO₄+SiO₂
substrate+BaSO₄+SnO₂+TiO₂+SiO₂
substrate+Al₂O₃+BaSO₄+SnO₂+SiO₂
substrate+ZnO+Al₂O₃+BaSO₄+SiO₂
substrate+Al₂O₃+BaSO₄+SnO₂
substrate+ZnO+Al₂O₃+BaSO₄

The filler pigments are prepared in the way that the substrate particles or mixtures of substrate particles are suspended in water, and a hydrolysable barium salt respectively, at least two different metal salt solutions or a metal salt and a silicate solution, preferably a sodium silicate solution, are added at a pH which is suitable for hydrolysis and which is selected in such a way that the barium sulfate and the metal oxides or metal oxide hydroxides are precipitated directly onto the substrates without secondary precipitations occurring. The pH is usually kept constant by simultaneous metered addition of a base and/or acid. The pigments are subsequently separated off, washed and in general dried at 50-150° C. for 6-18 h and optionally calcined for 5-120 minutes, where the calcination temperature can be optimised with respect to the coating present in each case. In general, the calcination temperatures are between 500 and 1000° C., preferably between 600 and 900° C. If desired, the pigments can be separated off, dried and optionally calcined after application of the barium sulfate coating and then resuspended for precipitation of the further metal oxide layer.

For preparing the barium sulfate layer a solution containing barium ions, all water-soluble barium salts, such as, for example, barium chloride, barium hydroxide, barium nitrate, can be used. Because of its advantageous price, ready availability and high purity, barium chloride is preferred. For preparing the solution containing sulfate ions, all soluble sulfates, such as, for example, titanium oxysulfate, sodium sulfate, potassium sulfate, magnesium sulfate, sodium bisulfate or potassium bisulfate, as well as sulfuric acid can be used.

The metal sulfates of the metals that form the metal oxides are highly soluble under the reaction conditions, and the metal oxides are precipitated under these conditions. Contrary to most of the metal oxides, barium oxide is soluble and BaSO₄ is highly insoluble under precipitation conditions; the solubility of the different metal oxides and metal sulfates enables achieving defined precipitations of BaSO₄ and other metal oxides.

The quantities in which the reactants are employed are not in themselves particularly critical. In particular, the quantity of the barium sulfate precipitated onto the substrate or metal oxide layer can be varied within wide limits, depending on the desired properties with regard to adhesive strength, extension and transparency on the skin.

Preferably, the starting suspensions contain about 5-10 weight percent of the flaky substrate, and the sulfate solution and the barium salt solution are added thereto as aqueous solutions containing about 5-25 percent by weight of the respective salt. Preferably, the barium salt is used in a quantity of 0.8-0.98 Ba²⁺ mol per mol of sulfate. After coating with the barium sulfate, the pigment is usually separated off, washed with water and dried.

In the next step a metal oxide or metal hydroxide is also precipitated on, in addition to the barium sulfate layer. The metal-oxide layers are preferably applied by wet-chemical methods, which have been developed for the preparation of pearlescent pigments. Methods of this type are described, for example, in DE 14 67 468, DE 19 59 988, DE 20 09 566, DE 22 14 545, DE 22 15 191, DE 22 44 298, DE 23 13 331, DE 15 22 572, DE 31 37 808, DE 31 37 809, DE 31 51 343, DE 31 51 354, DE 31 51 355, DE 32 11 602, DE 32 35 017 and in further patent documents and other publications known to the person skilled in the art.

As an alternative to the step-wise preparation described above the filler powder can also be prepared by simultaneous or subsequent precipitation of the barium sulfate and the metal salts in a one-pot-process.

If desired, the pigments formed can also be colored with colorants, and they can be very advantageously used together with the additives, conventional for this purpose, in face powders and similar preparations.

By using the foregoing procedures or equivalent ones, the desired firmly adhering coating will be achieved, i.e., the coating will remain on the substrate throughout the conventional use lifetime of the pigments. By small amounts of colored metal oxides a shade of color can be imparted to the pigments, which might be attractive for special uses.

In order to increase the light, water and weather stability, it is frequently advisable to subject the finished filler pigment, depending on the area of use, to post-coating or post-treatment. Suitable post-coatings or post-treatments are, for example, the processes described in German Patent 22 15 191, DE-A 31 51 354, DE-A 32 35 017 or DE-A 33 34 598. This post-coating further increases the chemical and photochemical stability or simplifies handling of the pigment, in particular incorporation into various media. In order to improve the wetability, dispersibility and/or compatibility with the user media, it is possible to apply, for example, functional coatings of Al₂O₃ or ZrO₂ or mixtures thereof to the pigment surface. Also possible are organic post-coatings, for example with silanes, as described, for example, in EP 0 090259, EP 0 634 459, WO 99/57204, WO 96/32446, WO 99/57204, U.S. Pat. No. 5,759,255, U.S. Pat. No. 5,571,851, WO 01/92425 or in J. J. Ponjeé, Philips Technical Review, Vol. 44, No. 3, 81 ff. and P. H. Harding J. C. Berg, J. Adhesion Sci. Technol. Vol. 11 No. 4, pp. 471-493.

The filler pigments according to the invention are versatile and can be employed in many areas. Accordingly, the present invention likewise relates to the use of the filler pigments according to the invention in cosmetics, paints, inks, printing inks, coatings, plastics, films, for the preparation of free-flowing pigment preparations and dry preparations like granules, pellets, etc.

In the case of cosmetics, the filler pigments according to the invention are particularly suitable for products and formulations in skin care products and anti-wrinkle products like make-ups, powders, loose powders, etc.

The concentration of the filler pigments in the application system to be pigmented is generally from 0.5 to 95% by weight, preferably 1 to 80% by weight and in particular 5 to 70% by weight. It is generally dependent on the specific application and can be up to 90% the case of loose powders. No limits are set for the concentrations of the filler pigments according to the invention in the formulation.

Preferably,
emulsions contain 0.1-30% by weight, in particular 1-15% by weight,
pigment-containing emulsions comprise 0.1-50% by weight, in particular 1-15% by weight, depending on the texture,
toothpastes contain 0.1-60% by weight, in particular 1-50% by weight,
water-free oil/wax-based products comprise 0.1-75% by weight, in particular 0.5-65% by weight,
powder products contain 0.1-95% by weight, in particular 1-75% by weight,
   of the filler pigments according to the invention, based on the formulation as a whole.

The filler pigments according to the invention can advantageously be employed in both decorative and care cosmetics.

The filler pigments can furthermore be mixed with commercially available state-of-the-art fillers. Fillers which may be mentioned are, for example, natural and synthetic mica, glass beads or glass powder, nylon powder, polymethylmethacrylate powders, pure or filled melamine resins, talc, glasses, kaolin, oxides or hydroxides of aluminium, magnesium, calcium or zinc, BiOCl, barium sulfate, calcium sulfate, calcium carbonate, magnesium carbonate, carbon, boron nitride and physical or chemical combinations of these substances. There are no restrictions regarding the particle shape of the filler. In accordance with requirements, it can be, for example, flake-form, spherical, needle-shaped, crystalline or amorphous.

The filler pigments according to the invention can of course also be combined in the formulations with cosmetic raw materials and auxiliaries of any type. These include, inter alia, oils, fats, waxes, film formers, surfactants, antioxidants, such as, for example, vitamin C or vitamin E, stabilisers, odour intensifiers, silicone oils, emulsifiers, solvents, such as, for example, ethanol, or ethyl acetate or butyl acetate, preservatives and auxiliaries which generally determine applicational properties, such as, for example, thickeners and rheological additives, such as, for example, bentonites, hectorites, silicon dioxides, Ca silicates, gelatines, high-molecular-weight carbohydrates and/or surface-active auxiliaries, etc.

The formulations comprising the pigment mixtures according to the invention can belong to the lipophilic, hydrophilic or hydrophobic type. In the case of heterogeneous formulations having discrete aqueous and non-aqueous phases, the pigment mixtures according to the invention may in each case be present in only one of the two phases or alternatively distributed over both phases.

The pH of the formulations can be between 1 and 14, preferably between 2 and 11 and particularly preferably between 5 and 8.

The pigments according to the invention may furthermore also be combined with cosmetic active ingredients. Suitable active ingredients are, for example, insect repellents, inorganic UV filters, such as, for example, TiO₂, UV A/BC protective filters (for example OMC, B3 and MBC), also in encapsulated form, anti-ageing active ingredients, vitamins and derivatives thereof (for example vitamin A, C, E, etc.), self-tanning agents (for example DHA, erythrulose, inter alia), and further cosmetic active ingredients, such as, for example, bisabolol, LPO, VTA, ectoine, emblica, allantoin, bioflavonoids and derivatives thereof.

Organic UV filters are generally incorporated into cosmetic formulations in an amount of 0.5 to 10% by weight, preferably 1 to 8%, and inorganic filters in an amount of 0.1 to 30%.

The preparations according to the invention may in addition comprise further conventional skin-protecting or skin-care active ingredients. These may in principle be any active ingredients known to the person skilled in the art.

Particularly preferred active ingredients are pyrimidine carboxylic acids and/or aryl oximes.

Of the cosmetic applications, particular mention should be made of the use of ectoine and ectoine derivatives for the care of aged, dry or irritated skin. Thus, European patent application EP-A-0 671 161 describes, in particular, that ectoine and hydroxyectoine are employed in cosmetic preparations, such as powders, soaps, surfactant-containing cleansing products, lipsticks, rouge, make-up, care creams and sunscreen compositions.

Application forms of the cosmetic formulations which may be mentioned are, for example: solutions, suspensions, emulsions, PIT (phase inversion temperature) emulsions, pastes, ointments, gels, creams, lotions, powders, soaps, surfactant-containing cleansing compositions, oils, aerosols and sprays. Examples of other application forms are sticks, shampoos and shower preparations. Any desired customary excipients, auxiliaries and, if desired, further active ingredients may be added to the preparation.

Ointments, pastes, creams and gels may comprise the customary excipients, for example animal and vegetable fats, waxes, paraffins, starch, tragacanth, cellulose derivatives, polyethylene glycols, silicones, bentonites, silica, talc and zinc oxide, or mixtures of these substances. Powders and sprays may comprise the customary excipients, for example lactose, talc, silica, aluminium hydroxide, calcium silicate and polyamide powder, or mixtures of these substances. Sprays may additionally comprise the customary propellants, for example chlorofluorocarbons, propane/butane or dimethyl ether.

Solutions and emulsions may comprise the customary excipients, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

Suspensions may comprise the customary excipients, such as liquid diluents, for example water, ethanol or propylene glycol, suspending agents, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the customary excipients, such as alkali metal salts of fatty acids, salts of fatty acid monoesters, fatty acid protein hydrolysates, isothionates, lanolin, fatty alcohol, vegetable oils, plant extracts, glycerol, sugars, or mixtures of these substances.

Surfactant-containing cleansing products may comprise the customary excipients, such as salts of fatty alcohol sulfates, fatty alcohol ether sulfates, sulfosuccinic acid monoesters, fatty acid protein hydrolysates, isothionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the customary excipients, such as synthetic oils, such as, for example, fatty acid esters, fatty alcohols, silicone oils, natural oils, such as vegetable oils and oily plant extracts, paraffin oils, lanolin oils, or mixtures of these substances.

The cosmetic preparations may exist in various forms. Thus, they can be, for example, a solution, a water-free preparation, an emulsion or microemulsion of the water-in-oil (W/O) or oil-in-water (O/W) type, a multiple emulsion, for example of the water-in-oil-in-water (W/O/W) type, a gel, a solid stick, an ointment or an aerosol. It is also advantageous to administer ectoines in encapsulated form, for example in collagen matrices and other conventional encapsulation materials, for example as cellulose encapsulations, in gelatine, wax matrices or liposomally encapsulated, In particular, wax matrices, as described in DE-A 43 08 282, have proven favourable. Preference is given to emulsions. O/W emulsions are particularly preferred. Emulsions, W/O emulsions and O/W emulsions are obtainable in a conventional manner.

Further embodiments are oily lotions based on natural or synthetic oils and waxes, lanolin, fatty acid esters, in particular triglycerides of fatty acids, or oily-alcoholic lotions based on a lower alcohol, such as ethanol, or a glycerol, such as propylene glycol, and/or a polyol, such as glycerol, and oils, waxes and fatty acid esters, such as triglycerides of fatty acids.

Solid sticks consist of natural or synthetic waxes and oils, fatty alcohols, fatty acids, fatty acid esters, lanolin and other fatty substances.

If a preparation is formulated as an aerosol, the customary propellants, such as alkanes, fluoroalkanes and chlorofluoroalkanes, are generally used.

Cosmetic formulations having light-protection properties may comprise adjuvants, such as surfactants, thickeners, polymers, softeners, preservatives, foam stabilisers, electrolytes, organic solvents, silicone derivatives, oils, waxes, antigrease agents, dyes and/or pigments which colour the composition itself or the hair, or other ingredients usually used in the cosmetic field.

The invention thus furthermore also relates to formulations comprising the filler pigment according to the invention in combination with at least one constituent selected from the group of absorbents, astringents, antimicrobial substances, antioxidants, antiperspirants, antifoaming agents, antidandruff active ingredients, antistatics, binders, biological additives, bleaching agents, chelating agents, deodorants, emollients, emulsifiers, emulsion stabilisers, dyes, humectants, film formers, fillers, odour substances, flavour substances, insect repellents, preservatives, anticorrosion agents, cosmetic oils, solvents, oxidants, vegetable constituents, buffer substances, reducing agents, surfactants, propellant gases, opacifiers, UV filters and UV absorbers, denaturing agents, viscosity regulators, perfume and vitamins.

In the case of the use of the filler pigments in paints and coatings, all areas of application known to the person skilled in the art are possible, such as, for example, powder coatings, printing inks for gravure, offset, screen or flexographic printing, toners and for coatings in outdoor applications. The paints and coatings here can be, for example, radiation-curing, physically drying or chemically curing. A multiplicity of binders is suitable for the preparation of printing inks or liquid surface coatings, for example based on acreages, methacrylates, polyesters, polyurethanes, nitrocellulose, ethylcellulose, polyamide, polyvinyl butyrate, phenolic resins, maleic resins, starch or polyvinyl alcohol, amino resins, alkyd resins, epoxy resins, polytetrafluoroethylene, polyvinylidene fluorides, polyvinyl chloride or mixtures thereof, in particular water-soluble grades. The surface coatings can be powder coatings or water- or solvent-based coatings, where the choice of the coating constituents is part of the general knowledge of the person skilled in the art. Common polymeric binders for powder coatings are, for example, polyesters, epoxides, polyurethanes, acrylates or mixtures thereof.

In addition, the filler pigments according to the invention can be used in films and plastics, gift foils, plastic containers and mouldings for all applications known to the person skilled in the art. Suitable plastics for the incorporation of the filler pigments according to the invention are all common plastics, for example thermosets or thermoplastics. The description of the possible applications and the plastics which can be employed, processing methods and additives are given, for example, in RD 472005 or in R. Glausch, M. Kieser, R. Maisch, G. Pfaff, J. Weitzel, Perlglanzpigmente [Pearlescent Pigments], Curt R. Vincentz Verlag, 1996, 83 ff., the disclosure content of which is also incorporated herein.

The filler pigments according to the invention are likewise suitable in the above-mentioned areas of application for use in blends with organic dyes and/or pigments, such as, for example, transparent and opaque white, coloured and black pigments, and with flake-form iron oxides, BiOCl, organic pigments, holographic pigments, LCPs (liquid crystal polymers) and conventional transparent, coloured and black lustre pigments based on metal oxide-coated flakes based on mica, glass, Al₂O₃, Fe₂O₃, SiO₂, metal flakes, etc. The filler pigments according to the invention can be mixed in any ratio with commercially available pigments and fillers.

The filler pigments according to the invention are furthermore suitable for the preparation of flowable pigment compositions and dry preparations comprising one or more particles according to the invention, binders and optionally one or more additives. Dry preparations is also taken to mean preparations which comprise from 0 to 8% by weight, preferably from 2 to 8% by weight, in particular from 3 to 6% by weight, of water and/or a solvent or solvent mixture. The dry preparations are preferably in the form of pellets, granules, chips, sausages or briquettes and have particle sizes of 0.2-80 mm. The dry preparations are used, in particular, in the preparation of printing inks and in cosmetic formulations.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentage are by weight, unless otherwise indicated.

### EXAMPLES

### Example

### Mica+BaSO₄+SnO₂+TiO₂

41.9 g BaCl₂ are dissolved in a suspension of 100 g muscovite mica (<15 μm) in 1400 g of deionized water and heated up to 75° C. while stirring. Subsequently, 280 g of a sodium sulfate solution (10 wt.-% of Na₂SO₄) are added with a dosing rate of 5 ml/minute.

After adjusting the pH to 1.8 by addition of hydrochloric acid (10 wt.-% of HCl) a solution of 11.7 g hydrochloric acid (37 wt.-% of HCl) and 18 g tin chloride solution (50 wt.-% of SnCl₄) in 296 g deionized water is added during 60 minutes. Subsequently, 371 g of titanium tetrachloride solution (32 wt.-% TiCl₄) are added with a dosing rate of 1.5 ml/min. During the addition of the tin chloride and titanium chloride solutions the pH is maintained by simultaneous addition of sodium hydroxide solution (32 wt.-% NaOH). After the addition of the titanium chloride solution the pH is elevated to 5.0 with sodium hydroxide solution and stirred for more 15 min.

For work up the product is isolated by filtration and washing with 10 l of deionized water and drying at 110° C. for 12 hours. Finally the product is calcined at 850° C. and sieved through a 40 μm sieve.

The obtained cosmetic filler exhibits bright powder color and an excellent skin feeling.

### Use Examples

### Use Example

### Face Powder

| Ingredients | | INCI | % |
|---|---|---|---|
| Phase A | | | |
| Silk Mica | (1) | MICA | 8.00 |
| Filler pigment according | (1) | | 8.00 |
| to Example 1 | | | |
| Ronasphere ® LDP | (1) | SILICA, CI 77891 | 5.00 |
| | | (TITANIUM DIOXIDE), CI | |
| | | 77491 (IRON OXIDES) | |
| Eusolex ® T-S | (1) | TITANIUM DIOXIDE, | 5.00 |
| | | ALUMINA, STEARIC ACID | |
| Unipure Yellow LC 182 | (2) | CI 77492 (IRON OXIDES) | 1.20 |
| Unipure Red LC 381 | (2 | CI 77491 (IRON OXIDES) | 0.20 |
| Unipure Brown LC 889 | (2) | CI 77491 (IRON OXIDES), | 0.30 |
| | | CI 77499 (IRON OXIDES) | |
| Magnesium stearate | (1) | MAGNESIUM STEARATE | 2.00 |
| Talc | (1) | TALC | 65.90 |
| Phase B | | | |
| RonaCare ® Tocopherol | (1) | TOCOPHERYL ACETATE | 0.30 |
| Acetate | | | |
| Fragrance 200 529 | (3) | PARFUM | 0.30 |
| Eutanol G | (4) | OCTYLDODECANOL | 3.70 |
| Propyl-4- | (1) | PROPYLPARABEN | 0.10 |
| hydroxybenzoate | | | |

### Procedure:

Grind the ingredients of phase A until the blend is uniform. Then add the pre-dissolved phase B and grind again until the whole phase A/B is uniform. Fill the bulk into pans and press with the desired pressure. The pressure for pans with 36 mm diameter is approx. 25 bar.

The obtained face powder is a light and transparent formula. Silk Mica delivers more smoothness to the texture while the filler pigment according to Example 1 and the Ronasphere® LDP are responsible for a subtle mattifying and light reflecting effect.

### Suppliers:

### (1) Merck KGaA/Rona®

### (2) Les Colorants Wackherr

### (3) Fragrance Resources

### (4) Cognis GmbH

The entire disclosure[s] of all applications, patents and publications, cited herein and of corresponding European patent application No. 07024443.9, filed Dec. 17, 2007, is incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usage and conditions.

1. A filler pigment comprising a platelet-shaped substrate coated with barium sulfate and at least two metal oxides and/or metal hydroxides.

2. A filler pigment according to claim 1, wherein the substrate is natural or synthetic mica, doped or undoped Al₂O₃ flakes, doped or undoped SiO₂ flakes, talc, kaolin, or doped or undoped glass flakes or mixtures thereof.

3. The filler pigment according to claim 1, wherein the metal oxides and/or metal hydroxides are TiO₂, SnO₂, ZnO, Fe₂O₃, Fe₃O₄, SiO₂, Al2O₃ or ZrO₂.

4. The filler pigment according to claim 1, wherein the substrate has an aspect ratio of 2 to 2000.

5. The filler pigment according to claim 1, containing an amount of barium sulfate of 5-200 wt.-% based on the substrate.

6. The filler pigment according to claim 1, having the following layer structure:

| | |
|---|---|
| substrate | |
| 1^{st} layer: | BaSO₄ |
| 2^{nd} layer: | metal oxide 1 |
| 3^{rd} layer: | metal oxide 2 or |
| substrate | |
| 1^{st} layer: | metal oxide 1 |
| 2^{nd} layer: | BaSO₄ |
| 3^{rd} layer: | metal oxide 2 or |
| substrate | |
| 1^{st} layer: | metal oxide 1 |
| 2^{nd} layer: | metal oxide 2 |
| 3^{rd} layer: | BaSO₄ or |
| substrate | |
| 1^{st} layer: | mixture of BaSO₄ + metal oxide 1 |
| 2^{nd} layer: | metal oxide 2 or |
| substrate | |
| 1^{st} layer: | metal oxide 1 |
| 2^{nd} layer: | mixture of BaSO₄ + metal oxide 2 or |
| substrate | mixture of BaSO₄, metal oxide 1 and metal oxide 2, |
| layer: | wherein metal oxide 1 and metal oxide 2 represent metal |
| | oxide layers which can be the same or different. |

7. The filler pigment according to claim 1, surface coated with a layer of SiO₂.

8. The filler pigment according to claim 1, having the following layer structure:
substrate+SnO₂+TiO₂+BaSO₄
substrate+mixture of SnO₂+TiO₂+BaSO₄
substrate+mixture of SnO₂+TiO₂+BaSO₄+layer of SiO₂ on top
substrate+SiO₂+TiO₂+BaSO₄
substrate+SiO₂+SnO₂+BaSO₄
substrate+SnO₂+TiO₂+BaSO₄+Carmine red
substrate+SnO₂+TiO₂+BaSO₄+Prussian Blue
substrate+SnO₂+BaSO₄/TiO₂
substrate+SnO₂+TiO₂+BaSO₄+SiO₂
substrate+BaSO₄+SnO₂+TiO₂+SiO₂
substrate+Al₂O₃+BaSO₄+SnO₂+SiO₂
substrate+ZnO+Al₂O₃+BaSO₄+SiO₂
substrate+Al₂O₃+BaSO₄+SnO₂
substrate+ZnO+Al₂O₃+BaSO₄

9. A process for the production of a filler pigment of claim 1, comprising suspending the substrate in an aqueous solution, adding the barium salt and at least two metal salt solutions at a pH which is suitable for hydrolysis and which is selected in such a way that the barium sulfate and the metal oxides and/or hydroxides are precipitated directly onto the substrate.

10. In paint, lacquer, ink, printing ink, plastic or cosmetic formulation comprising a filler pigment, the improvement wherein the filler pigment is one according to claim 1.

11. A cosmetic formulation containing a filler pigment of claim 1, in an amount of up to 95% by weight.

12. The cosmetic formulation according to claim 11, further comprising at least one absorbent, astringent, antimicrobial substance, antioxidant, antiperspirant, antifoaming agent, antidandruff active ingredient, antistatic, binder, biological additive, bleaching agent, chelating agent, deodorant, emollient, emulsifier, emulsion stabilizer, dye, humectant, film former, filler, odor imparting substance, flavorant, insect repellent, preservative, anticorrosion agent, cosmetic oil, solvent, oxidant, vegetable constituent, buffer substance, reducing agent, surfactant, propellant gas, opacifier, UV filter and UV absorber, denaturing agent, viscosity regulator, perfume or vitamin.

13. A filler pigment comprising platelet-shaped natural or synthetic mica coated with barium sulfate and at least two metal oxides and/or metal hydroxides.

14. A filler pigment comprising doped or undoped glass flakes or mixtures thereof coated with barium sulfate and at least two metal oxides and/or metal hydroxides.

15. In paint, lacquer, ink, printing ink, plastic or cosmetic formulation comprising a filler pigment, the improvement wherein the filler pigment is one according to claim 13.

16. A cosmetic formulation containing a filler pigment of claim 13, in an amount of up to 95% by weight.

17. The cosmetic formulation according to claim 16, further comprising at least one absorbent, astringent, antimicrobial substance, antioxidant, antiperspirant, antifoaming agent, antidandruff active ingredient, antistatic, binder, biological additive, bleaching agent, chelating agent, deodorant, emollient, emulsifier, emulsion stabilizer, dye, humectant, film former, filler, odor imparting substance, flavorant, insect repellent, preservative, anticorrosion agent, cosmetic oil, solvent, oxidant, vegetable constituent, buffer substance, reducing agent, surfactant, propellant gas, opacifier, UV filter and UV absorber, denaturing agent, viscosity regulator, perfume or vitamin.

18. In paint, lacquer, ink, printing ink, plastic or cosmetic formulation comprising a filler pigment, the improvement wherein the filler pigment is one according to claim 14.

19. A cosmetic formulation containing a filler pigment of claim 14, in an amount of up to 95% by weight.

20. The cosmetic formulation according to claim 19, further comprising at least one absorbent, astringent, antimicrobial substance, antioxidant, antiperspirant, antifoaming agent, antidandruff active ingredient, antistatic, binder, biological additive, bleaching agent, chelating agent, deodorant, emollient, emulsifier, emulsion stabilizer, dye, humectant, film former, filler, odor imparting substance, flavorant, insect repellent, preservative, anticorrosion agent, cosmetic oil, solvent, oxidant, vegetable constituent, buffer substance, reducing agent, surfactant, propellant gas, opacifier, UV filter and UV absorber, denaturing agent, viscosity regulator, perfume or vitamin.
